(19) United States
(12) Patent Application Publication
(10) Pub. No.: US 2005/0277892 A1
(43) Pub. Date: Dec. 15, 2005

(54) Leakproof needle cover structure improvement

(75) Inventor: Tung-Hao Chen, Taipei (TW)

Correspondence Address: BACON & THOMAS, PLLC, 625 SLATERS LANE, FOURTH FLOOR, ALEXANDRIA, VA, 22314, US

(73) Assignee: Feng-Chi Tsao, Hsinchu (TW)

(21) Appl. No.: 10/863,263

(22) Filed: Jun. 9, 2004

(51) Int. Cl. A61M 5/32

(52) U.S. Cl. 604/192; 604/263

(57) ABSTRACT

The present invention is a leakproof needle cover structure improvement. It relates to a safety hypodermic syringe with the needle and the needle holder retractable to the syringe, in which the needle cover has a cap at its end part, and leakage-proof ring is formed around the inside of the cap, such that when the needle cover sheathing on the needle retracts, the cap will simultaneously cover the aperture of the barrel, thus effectively prevent the leakage of residual medicament kept in the barrel.

### BACKGROUND OF THE INVENTION

1. Field of the Invention

The present invention relates to a leakproof needle cover structure improvement and, more specifically, to a needle cover with a cap provided at the front end of the said needle cover, in which the outer diameter of the said cap is broader than the aperture of the safety barrel such that when the needle and the needle holder are locked and pulled back by the plunger, the cap will cover the aperture of the barrel, the leakage of residual medicament kept in the barrel is accordingly prevented.

2. Description of Related Art

For medical employees, medical infections are mostly caused by inadequate use of medical apparatus. Take the hypodermic syringe as an example, the syringe needle is an apparatus having direct contact with the patient, it thus can only be used once. However, medical employees are frequently stung by used needle because of incaution or inexperience, which certainly causes transmitted infection. Consequently, safety hypodermic syringe is introduced to ensure the syringe is used once, and the function of retracting needle into the barrel is provided to protect medical employees from infections caused by stab of the syringe needle.

The said safety hypodermic syringe, referring to FIG. 1, has a needle holder 5 and a plunger 4 in the hollow barrel 3, where the center of the retainer tip 41 located at the front end of plunger 4 provides a locker 42. Therefore, when the plunger 4 is push forwards to the bottom, locker 42 can lock needle holder 5 and interlock needle 2. In contrast, when the plunger 4 is pulled backwards, the needle holder 5 together with needle 2 are pulled back into the chamber 31 of the hollow barrel 3, and after the plunger 4 is broken, the needle 2 is kept inside the barrel 3 from stinging. As a result, the barrel 3 is not reusable and the recycling of syringe is effectively prohibited.

While that safety hypodermic syringe is an invention benefiting medical employees, the inventor observed that, after use, the action of fitting the needle cover 1 for the needle 2 is overlooked. That is, although the plunger 4 is broken such that the needle holder 5 and needle 2 can not be pushed forwards after the operation, it is possible that the needle holder 5 and needle 2 may further pushed forwards by a rod or other items with size smaller than the inner diameter of barrel 3 after the syringe was abandoned, the personnel engaged in the medical waste may therefore be pricked.

Notwithstanding the existing needle cover 1 were sheathed onto the needle 2, little medicament will inevitably be kept inside the hollow barrel 3 because of the retraction of the needle holder 5 and needle 2, which will cause medicament leakage after a brief upside-down dislocation. Accordingly, the problem of medicament leakage is not solvable by simply sheathing needle cover 1 onto needle 2. As the aperture 32 of the barrel 3 is kept open, only sealing the aperture 32 of the barrel 3 can we avoid the sting caused by incautious disclosure of needle 2 and can we avoid the leakage of medicament.

### SUMMARY OF THE INVENTION

The main object of this leakproof needle cover structure improvement is to solve the aforementioned shortcomings. More specifically, the present invention is a needle cover providing a cap at its front end, where the outer diameter of the cap is broader than the aperture of the safety barrel. In that way, when the needle as well as the needle holder are locked and pulled backwards by the plunger, the aperture of barrel is simultaneously covered such that the barrel is completely closed and the needle together with the needle holder are sealed inside the hollow barrel. Accordingly, the incautious sting by disclosed needle and the leakage of residual medicament are avoided. Moreover, as the needle cover is placed inside the barrel, the problem of randomly discarding needle cover is eradicated.

To further demonstrate the features, objects, and effectiveness of the present invention, the preferred embodiments together with the accompanying drawings are described in the following.

### BRIEF DESCRIPTION OF THE DRAWINGS

FIG. 1 is an exploded view of the present invention embodied in a conventional safety hypodermic syringe.

FIG. 2 is a sectional view of the present invention.

FIG. 3 is a sectional view showing the action of the present invention (I).

FIG. 4 is a sectional view showing the action of the present invention (II).

FIG. 5 is a sectional view showing the action of another embodiment of the present invention.

### DETAILED DESCRIPTION OF THE PREFERRED EMBODIMENT

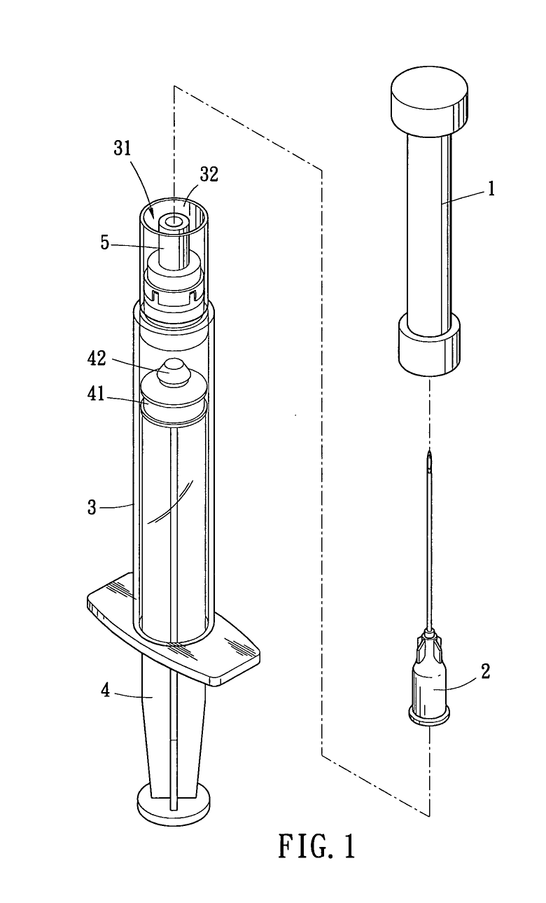

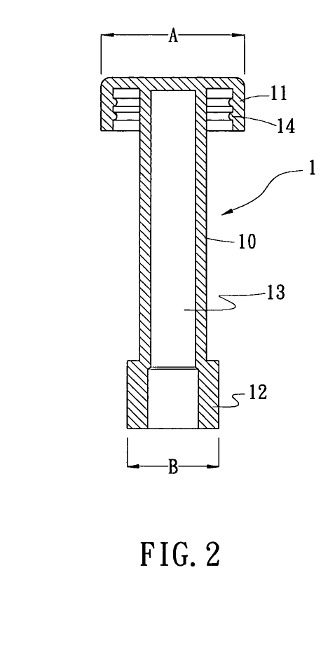

Firstly, referring to FIG. 1 and FIG. 2, to effectively avoid the sting caused by incautious disclosure of needle 2 or medicament leakage, we must completely seal the aperture 32 of the barrel 3. The present invention, the leakproof needle cover structure improvement, mainly is a needle cover 1 formed by injection molding, which provides a cap 11 at its front end, a tube 10, and a lower cap 12, where the lower cap 12 is a reception aperture to be inserted by needle 2, and the tube 10 is a hollow room 13 for allocating needle 2, while the outer diameter A of cap 11 is broader than the aperture 32 of barrel 3, and leakproof ring 14 is provided around the inside of the cap 11 such that the leakage is effectively avoided when the cover 11 is sheathed onto the aperture 32 of the barrel 3.

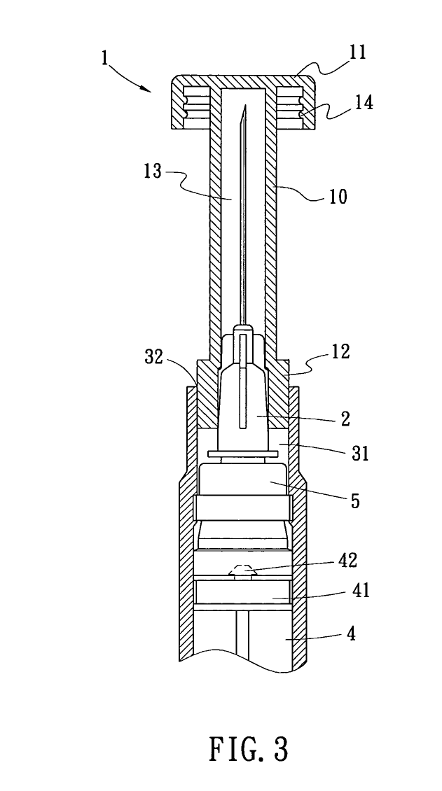

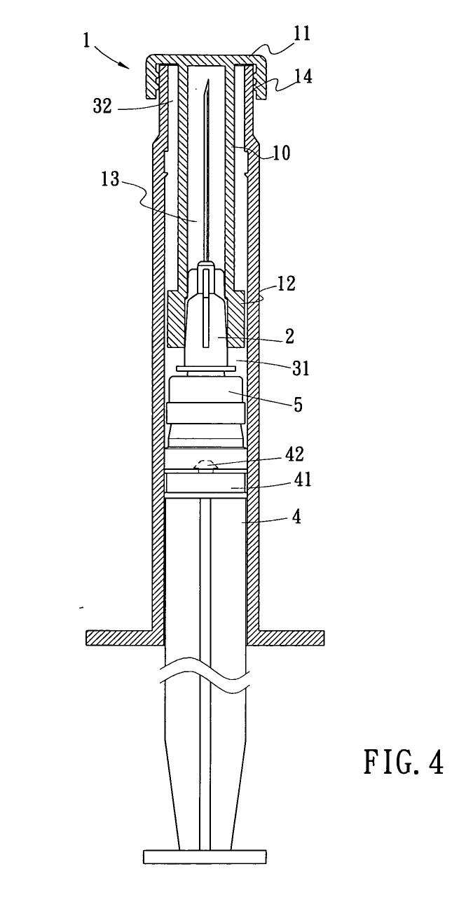

When the conventional safety hypodermic syringe is in use, referring to FIG. 3 and FIG. 4, the needle 2 is firstly inserted into needle holder 5. After injection is completed, needle cover 1 is sheathed onto the needle 2, in which the outer diameter B of the lower cap 12 of the needle cover 1 should be smaller than the inner diameter of the aperture 32 of the barrel 3. Then, as the locker 42 locks needle holder 5 and interlocks needle 2 when the plunger 4 is pushed to the bottom, the needle holder 5 and needle 2 will be pulled back into the chamber 31 of the hollow barrel 3 when the plunger 4 is pulled backwards, and, as shown in FIG. 4, the leakproof ring 14 inside the cap of the needle cover 1. will compactly cover the aperture 32 of the barrel 3 such that the barrel 3 is closed completely, and the needle holder 5 and needle 2 are completely sealed inside the hollow barrel 3. The sting caused by incautious disclosure of needle 2 is thus effectively avoided and the leakage of residual medicament kept inside the barrel 3 is also avoided.

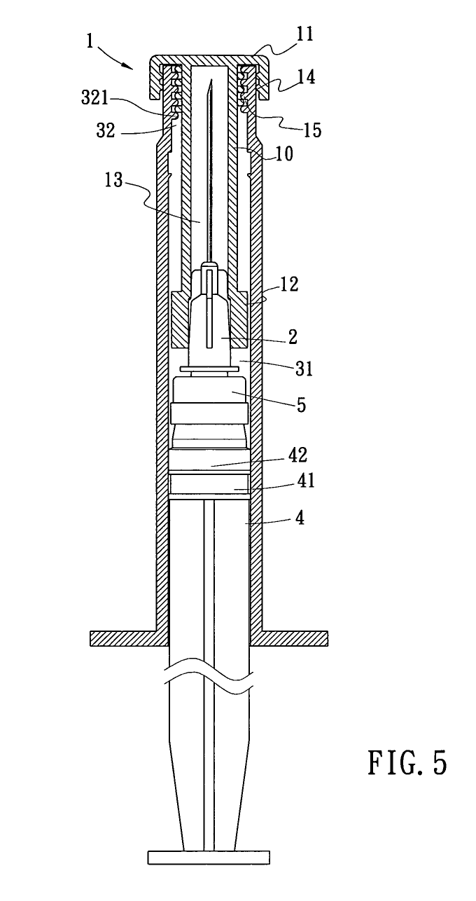

Moreover, referring to FIG. 5, another embodiment of the leakproof needle cover structure improvement, in which needle cover 1 can be embodied in a screw 15 structure inside the tube 10 of cap. For the screw part 321 inside the aperture 32 of barrel 3, as another barrel 3 structure, in which the aperture 321 provides screw part 321 such that the needle 2 can be screwed on the needle holder 5, is commercially available, another embodiment of the present invention can be applied to this type of barrel 3, such that the needle cover 1 can be screwed into the aperture 32 of barrel 3 through screw 15. In this way, the disengagement of needle cover 1 can be effectively prevented, and the coherence between needle cover 1 and barrel 3 is further assured.

1. A leakproof needle cover structure improvement, which is a needle cover for safety hypodermic syringe with the needle and the needle holder retractable into the syringe; the said needle cover is formed by injection molding, which provides a cap at its front end, a tube, and a lower cap, wherein the outer diameter of the said cap is broader than the aperture of barrel, such that when the needle and the needle holder is locked and pulled back by the plunger, the cover is simultaneously covered onto the aperture of the barrel, the leakage of residual medicament kept in barrel is thus prevented the main features of the leakproof needle cover structure improvement is that the outer diameter of the said cap is broader than the aperture of barrel, leakproof ring is provided around the inside of the cap, and after injection is completed, needle cover is sheathed onto the needle when the plunger is pulled backwards, the needle holder as well as the needle will be pulled back into the hollow barrel, and the cap of the needle cover will compactly cover the aperture of the barrel such that the leakage is effectively prevented.

2. A leakproof needle cover structure improvement as claimed in claim 1, wherein the outer diameter of the lower cap must be smaller than that of the barrel so as to pass through the aperture of the barrel during retracting.

3. A leakproof needle cover structure improvement, which is a needle cover for safety hypodermic syringe with the needle and the needle holder retractable into the syringe; the said needle cover is formed by injection molding, which provides a cap at its front end, a tube, and a lower cap, wherein the outer diameter of the said cap is broader than the aperture of barrel, such that when the needle and the needle holder is locked and pulled back by the plunger, the cover is simultaneously covered onto the aperture of the barrel, the leakage of residual medicament kept in barrel is thus prevented the main features of the leakproof needle cover structure improvement is that, the said needle cover can be embodied in a screw structure inside the tube of cap for commercially available barrel structure providing screw part inside the aperture, the screw structure of the tube can be screwed into the screw part inside the aperture of the barrel, the barrel and the needle cover are thus composed compactly.
